(19) United States
(12) Patent Application Publication
(10) Pub. No.: US 2025/0143731 A1
(43) Pub. Date: May 8, 2025

(54) TOOLS FOR BODILY IMPLANTS

(71) Applicant: Boston Scientific Scimed, Inc., Maple Grove, MN (US)

(72) Inventors: Subodh Morey, Ponda (IN); Arun Adhikarath Balan, Aluva (IN); Rajivkumar Singh, Thane (IN); Aditya Dhanotiya, Indore (IN)

(21) Appl. No.: 19/018,873

(22) Filed: Jan. 13, 2025

Related U.S. Application Data
(63) Continuation of application No. 17/247,384, filed on Dec. 9, 2020.
(60) Provisional application No. 62/947,219, filed on Dec. 12, 2019.

(51) Int. Cl. A61B 17/28; A61F 2/26; B25B 27/02

(52) U.S. Cl. CPC A61B 17/2804; A61F 2/26; B25B 27/02

(57) ABSTRACT

According to an aspect, an apparatus includes a handle portion having a first portion and a second portion, a distal portion disposed opposite the handle portion, the distal portion having a first connector contacting portion and a second connector contacting portion, the first connector contacting portion being configured to move with respect to the second connector contacting portion, the distal portion being configured to be disposed in a first configuration and a second configuration, the first connector contacting portion being disposed a first distance from the second connector contacting portion when the distal portion is in the first configuration, the first connector contacting portion being disposed a second distance from the second connector contacting portion when the distal portion is in the second configuration, the second distance being greater than the first distance, and a biasing member configured to bias the distal portion into the first configuration.

### CROSS-REFERENCE TO RELATED APPLICATIONS

This application is a continuation of, and claims priority to, U.S. patent application Ser. No. 17/247,384, filed on Dec. 9, 2020, entitled “TOOLS FOR BODILY IMPLANTS”, which claims priority to U.S. Provisional Patent Application No. 62/947,219, filed on Dec. 12, 2019, entitled “TOOLS FOR BODILY IMPLANTS”, the disclosures of which are incorporated by reference herein in their entirety.

### TECHNICAL FIELD

This disclosure relates generally to tools for placing bodily implants within a body of a patient and more specifically to tools that can be used to facilitate the placement of a tubing or a tubing portion of a bodily implant.

### BACKGROUND

Bodily implants that include tubes or tubular members are placed within the body to treat the body. For example, one treatment for male erectile dysfunction is the implantation of a penile prosthesis that mechanically erects the penis. Some such penile prostheses include tubes or tubular member. In some cases, the tubes or tubular members must be connected during the implantation process. In some procedures, a tool is used to facilitate the connection of such tubes or tubular members.

Accordingly, there is a need for tools that can be used to facilitate the connection of such tubes or tubular members.

### SUMMARY

According to an aspect, an apparatus includes a handle portion having a first portion and a second portion, a distal portion disposed opposite the handle portion, the distal portion having a first connector contacting portion and a second connector contacting portion, the first connector contacting portion being configured to move with respect to the second connector contacting portion, the distal portion being configured to be disposed in a first configuration and a second configuration, the first connector contacting portion being disposed a first distance from the second connector contacting portion when the distal portion is in the first configuration, the first connector contacting portion being disposed a second distance from the second connector contacting portion when the distal portion is in the second configuration, the second distance being greater than the first distance, and a biasing member configured to bias the distal portion into the first configuration.

In some embodiments, the biasing member is disposed between the first portion of the handle portion and the second portion of the handle portion. In some embodiments, the biasing member is a spring. In some embodiments, the biasing member is a spring, the spring being configured to engage the first portion of the handle portion and the second portion of the handle portion. In some embodiments, the biasing member is a living hinge.

In some embodiments, the first portion of the handle portion is configured to move with respect to the second portion of the handle portion. In some embodiments, the first portion of the handle portion being configured to move towards the second portion of the handle portion to place the distal portion in the second configuration.

In some embodiments, the first portion of the handle portion includes a stop member, the second portion of the handle portion includes a stop member, the stop member of the first handle portion being configured to contact the stop member of the second handle portion. In some embodiments, the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location.

In some embodiments, the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location, the first portion of the handle portion being coupled to the second portion of the handle portion. In some embodiments, the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location, the first portion of the handle portion being coupled to the second portion of the handle portion at a third location of the first portion of the handle portion, the third location being different than the first location, third location being different than the second location. In some embodiments, the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location, the first portion of the handle portion being coupled to the second portion of the handle portion at a third location of the first portion of the handle portion, the third location being disposed between the first location and the third location.

In some embodiments, the first portion of the handle portion includes a projection portion, the second portion of the handle portion includes a receiving portion configured to receive the projection portion of the first portion of the handle portion. In some embodiments, the first portion of the handle portion includes a projection portion, the second portion of the handle portion includes a receiving portion configured to receive the projection portion of the first portion of the handle portion, the second portion of the handle portion includes a projection portion, the second portion of the handle portion includes a projection portion, the first portion of the handle portion includes a receiving portion configured to receive the projection portion of the second portion of the handle portion.

In some embodiments, the first portion of the distal portion includes a receiving portion configured to receive a portion of a connector.

According to another aspect, an apparatus includes a handle portion having a first member and a second member, the first member of the handle portion being coupled to the second member of the handle portion at a first location; and a distal portion having a first member and a second member, the first member of the handle portion being coupled to the first member of the distal portion at a second location, the first member of the handle portion being coupled to the second member of the distal portion at a third location, the first location being disposed between the second location and the third location, the first member of the distal portion having an engagement portion configured to engage a connector.

In some embodiments, the apparatus includes a biasing member, the biasing member being disposed between the first member of the handle portion and the second member of the handle portion.

In some embodiments, the first member of the distal portion is configured to move with respect to the second member of the distal portion from a first configuration to a second configuration.

According to another aspect, an apparatus includes a first elongate member having a handle portion and a distal portion, the first elongate member having a projection; a second elongate member having a handle portion and a distal portion, the second elongate member having a receiving portion, the receiving portion being configured to receive the projection; and a biasing member disposed between the first elongate member and the second elongate member.

In some embodiments, the first elongate member is configured to move with respect to the second elongate member from a first configuration to a second configuration.

### BRIEF DESCRIPTION OF THE DRAWINGS

FIG. 1 schematically illustrates an apparatus according to an aspect.

FIG. 2 is a perspective view of an apparatus according to an aspect.

FIG. 3 is a side view of a portion of the apparatus of FIG. 2.

FIG. 4 is a perspective view of a portion of the apparatus of FIG. 2.

FIG. 5 is a side view of the apparatus of FIG. 2 in an open configuration.

FIG. 6 is a side view of the apparatus of FIG. 2 in a closed configuration.

FIGS. 7-9 illustrate a connection member of a bodily implant.

FIG. 10 is a perspective view of an apparatus according to an aspect.

FIG. 11 is a side view of the apparatus of FIG. 10 in a closed configuration.

FIG. 12 is a perspective view of the apparatus of FIG. 10.

FIG. 13 is a side view of the apparatus of FIG. 10 in an open configuration.

FIG. 14 is side view of an apparatus according to an aspect in a closed configuration.

FIG. 15 is a perspective view of the apparatus of FIG. 14.

FIG. 16 is a side view of the apparatus of FIG. 14 in an open configuration.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to tools or devices to facilitate the implantation or placement of bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure.

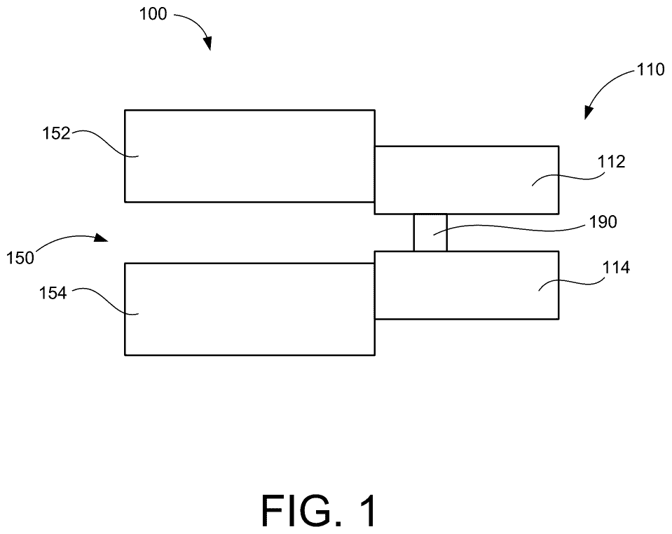

FIG. 1 schematically illustrates a tool 100 for implanting an implant into a body of a patient. In some embodiments, that tool 100 may be used to engage connectors that may be used to connect tubular member or tubes of a bodily implant. For example, in some embodiments, the tool 100 may be used to engage connectors used to connect tubular members of a penile implant such as a penile implant or a penile prosthesis.

The tool 100 includes a handle portion 110 and a distal portion 150. The handle portion 110 includes a first portion 112 and a second portion 114. In some embodiments, the first portion 112 of the handle portion 110 is a separate member from the second portion 114 of the handle portion 110. In other embodiments, the first portion 112 and the second portion 114 may be formed unitarily or monolithically.

The distal portion 150 includes a first portion 152 and a second portion 154. In some embodiments, the distal portion 150 is configured to engage a portion of a bodily implant. For example, in some embodiments, the distal end portion 150 is configured to engage connectors that are used to connect tubular members of a bodily implant.

The first portion 152 of the distal portion 150 is configured to move with respect to the second portion 154 of the distal portion 150. For example, in one embodiment, the first portion 152 is configured to move with respect to the second portion 154 from a first configuration (or open configuration) to a second configuration (or closed configuration). The distance between the first portion 152 and the second portion 154 in the first configuration is greater than the distance between the first portion 152 and the second portion 154 when in the second configuration.

In the illustrated embodiment, the tool 100 includes a biasing member 190. The biasing member 190 is configured to bias the tool 100 such that the distal portion 150 is biased into one of its configurations. For example, in some embodiments, the biasing member 190 is configured to bias the distal portion 150 to an open configuration. In some embodiments, the biasing member 190 is a spring member. In other embodiments, the biasing member 190 is another type of biasing device or member.

For example, in some embodiments, the biasing member 190 is a biased hinge such as a living hinge member.

In some embodiments, the first portion 112 of the handle portion 110 is configured to move with respect to the second portion 114 of the handle portion 110. Movement of the first portion 112 with respect to the second portion 114 causes the first portion 152 of the distal portion 150 to move with respect to the second portion 154 of the distal portion 150. For example, movement of the first portion 112 of the handle portion 110 with respect to the second portion 14 of the handle portion 110 may cause the distal portion 150 to move from the first configuration (open configuration) to the second configuration (closed configuration).

In some embodiments, the tool 100 is configured to be a single use device. In other words, in some embodiments, the tool 100 is configured to be used in a single procedure on a single patient and then discarded or disposed of.

In some embodiments, the tool 100 is formed of a biocompatible plastic material. For example, in some embodiments, the first portion of the handle portion, the second portion of the handle portion, the first portion of the distal portion, and the second portion of the distal portion are formed or made from a biocompatible plastic material. In other embodiments, the tool 100 is formed of other materials including other biocompatible materials.

In use, the tool 100 may be placed within a body of a patient. The tool 100 may be placed such that the first portion 152 of the distal portion 150 engages a first portion of a connector and the second portion 154 of the distal portion 150 engages a second portion of the connector. The first portion 112 of the handle portion 110 may then be moved with respect to the second portion 114 of the handle portion 110 to move the distal portion from an open configuration to a closed configuration. The movement moves the first portion of the connector towards the second portion of the connector to connect the connector and any tubular members coupled to the connectors. The user may then release the handle portion to return the distal portion to the open configuration (because of the bias member).

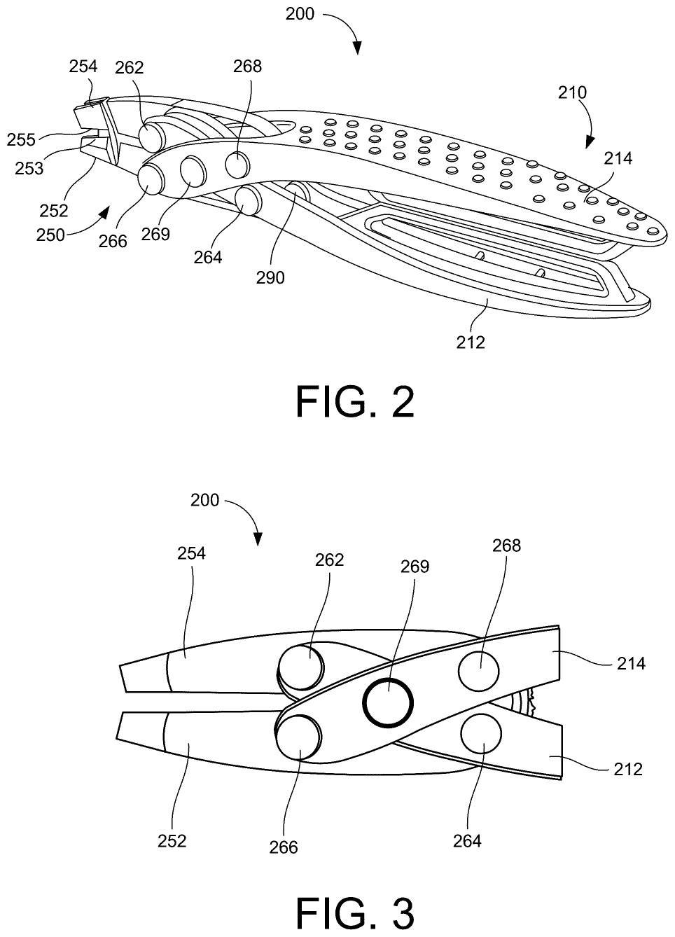

FIG. 2 is a perspective view of an apparatus 200 according to an aspect.

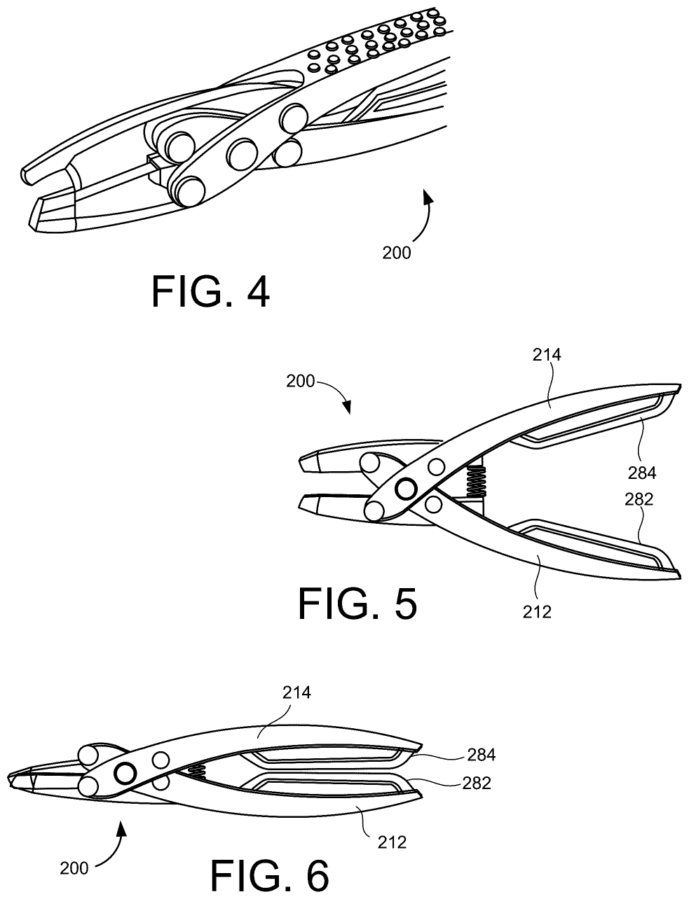

FIG. 3 is a side view of a portion of the apparatus 200. FIG. 4 is a perspective view of a portion of the apparatus 200. FIG. 5 is a side view of the apparatus 200 in an open configuration. FIG. 6 is a side view of the apparatus 200 in a closed configuration.

In some embodiments, that tool 200 may be used to engage connectors that may be used to connect tubular member or tubes of a bodily implant. For example, in some embodiments, the tool 200 may be used to engage connectors used to connect tubular members of a penile implant such as a penile implant or a penile prosthesis.

The tool 200 includes a handle portion 210 and a distal portion 250. The handle portion 210 includes a first portion or member 212 and a second portion or member 214. In the illustrated embodiment, the first portion 212 of the handle portion 210 is a separate member from the second portion 214 of the handle portion 210.

The distal portion 250 includes a first portion or member 252 and a second portion or member 254. In some embodiments, the distal portion 250 is configured to engage a portion of a bodily implant. For example, in some embodiments, the distal end portion 250 is configured to engage connectors or portions of a connector that are used to connect tubular members of a bodily implant. In the illustrated embodiment, the first portion or member 252 includes a connector engagement portion 253 and the second portion or member 254 includes a connector engagement portion 255.

The first portion or member 252 of the distal portion 250 is configured to move with respect to the second portion or member 254 of the distal portion 250. For example, in one embodiment, the first portion or member 252 is configured to move with respect to the second portion or member 254 from a first configuration (or open configuration) to a second configuration (or closed configuration). The distance between the first portion or member 252 and the second portion or member 254 in the first configuration is greater than the distance between the first portion or member 252 and the second portion or member 254 when in the second configuration.

In the illustrated embodiment, the tool 200 includes a biasing member 290. The biasing member 290 is configured to bias the tool 200 such that the distal portion 250 is biased into one of its configurations. For example, in the illustrated embodiment, the biasing member 290 is configured to bias the distal portion 250 to an open configuration. In other embodiments, the biasing member may be configured to bias the distal portion to a closed configuration.

In the illustrated embodiment, the biasing member 290 is a spring member. In other embodiments, the biasing member is another type of biasing device or member.

In the illustrated embodiment, the first portion or member 252 of the distal portion 250 is operatively coupled to the second portion or member 214 of the handle portion 210. For example, a pin or rivet type coupler 266 pivotally or rotatably couples the first portion or member 252 of the distal portion 250 to the second portion or member 214 of the distal portion 210 at a first location. The first portion or member 252 of the distal portion 250 is also coupled the first portion 212 of the handle portion 210 via a pin or a rivet type coupler 264 at a second location different than the first location.

Similarly, the second portion or member 254 of the distal portion 250 is operatively coupled to the first portion or member 212 of the handle portion 210. For example, a pin or rivet type coupler 262 pivotally or rotatably couples the second portion or member 254 of the distal portion 250 to the first portion or member 212 of the handle portion 210 at a third location. The second portion or member 254 of the distal portion 250 is also coupled the second portion 214 of the handle portion 210 via a pin or a rivet type coupler 268 at a fourth location different than the third location.

The first portion or member 212 of the handle portion 210 is operatively coupled to the second portion or member 214 of the handle portion 210. In the illustrated embodiment, the first portion or member 212 of the handle portion 210 is pivotally or rotatably coupled to the second portion or member 214 of the handle portion 210 via a pin or rivet type coupler 269 at a fifth location. In the illustrated embodiment, the fifth location is between the first location and the second location and is also between the third location and the fourth location.

In the illustrated embodiment, the first portion 212 of the handle portion 210 is configured to move with respect to the second portion 214 of the handle portion 210. Movement of the first portion 212 with respect to the second portion 214 causes the first portion 252 of the distal portion 250 to move with respect to the second portion 254 of the distal portion 250. For example, movement of the first portion 252 of the handle portion 250 with respect to the second portion 254 of the handle portion 250 may cause the distal portion 250 to move from the first configuration (open configuration) to the second configuration (closed configuration).

In the illustrated embodiment, the first portion or member 212 of the handle portion 210 includes a stop or contact portion 282. The second portion or member 214 of the handle portion 210 includes a stop or contact portion 284. The stop or contact portion 282 is configured to contact or engage the stop or contact portion 284 when the first portion or member 212 moves towards the second portion or member 214.

In some embodiments, the tool 200 is configured to be a single use device. In other words, in some embodiments, the tool 200 is configured to be used in a single procedure on a single patient and then discarded or disposed of.

In some embodiments, the tool 200 is formed of a biocompatible plastic material. For example, in some embodiments, the first portion of the handle portion, the second portion of the handle portion, the first portion of the distal portion, and the second portion of the distal portion are formed or made from a biocompatible plastic material. In other embodiments, the tool 200 is formed of other materials including other biocompatible materials.

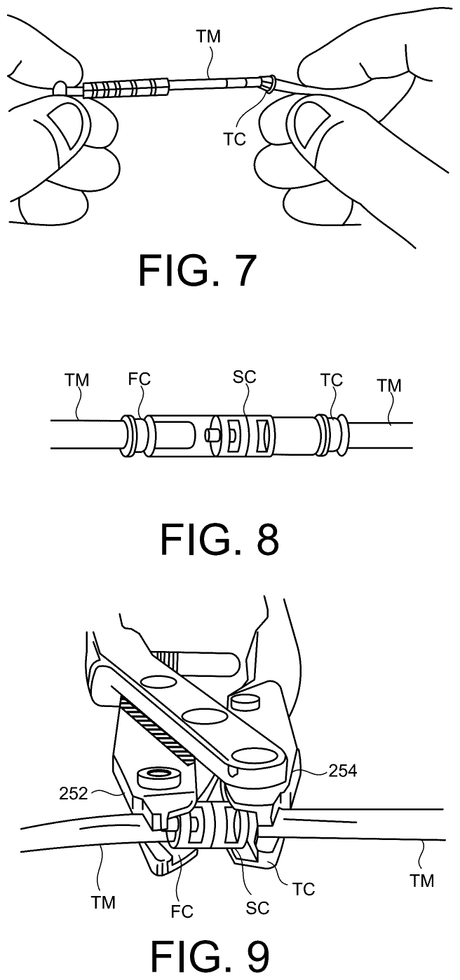

FIGS. 7-9 illustrate a connection member of a bodily implant and the use of the tool 200. As illustrated in FIGS. 7 and 8, a first connector member FC may be placed between two tubular members TM of a bodily implant. A second connector member SC may be placed over one of the tubular members and a third connector member TC may be placed over the other of the tubular members.

As illustrated in FIG. 9, the tool or apparatus 200 may be placed within a body of a patient. The tool or apparatus 200 may be placed such that the first portion 252 of the distal portion 250 engages the second connection member SC and the second portion 254 of the distal portion 250 engages the third connection member TC. The first portion 212 of the handle portion 210 may then be moved with respect to the second portion 214 of the handle portion 210 to move the distal portion from an open configuration to a closed configuration. The movement moves the second connector member SC towards the third connector member TC to couple the second connector member SC to the first connector member FC and the second connector member SC to the third connector member TC. Accordingly, the tubular members TM may be coupled together. The user may then release the handle portion to return the distal portion to the open configuration (for example, because of the bias member).

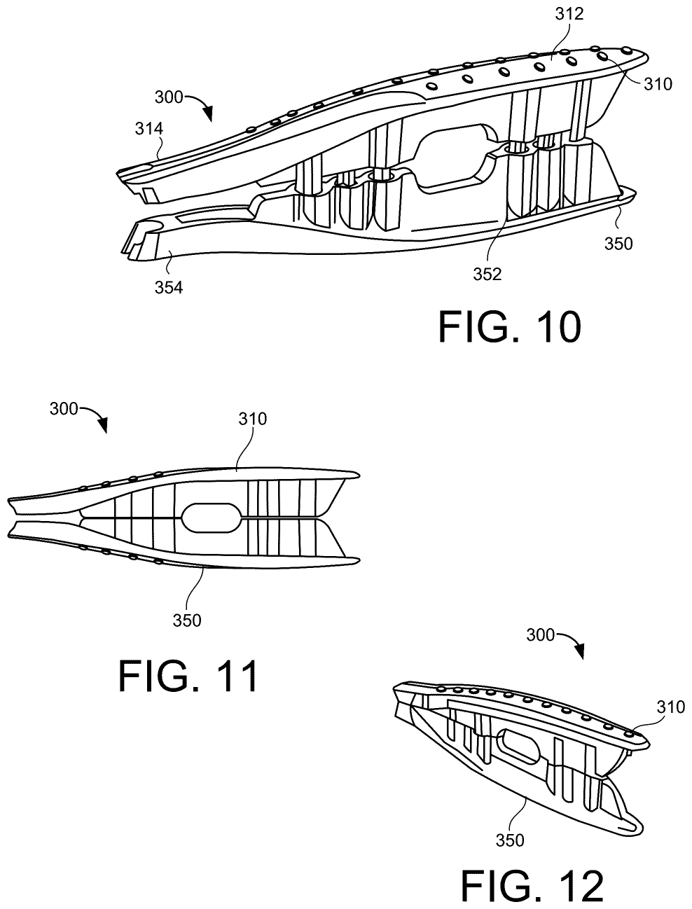

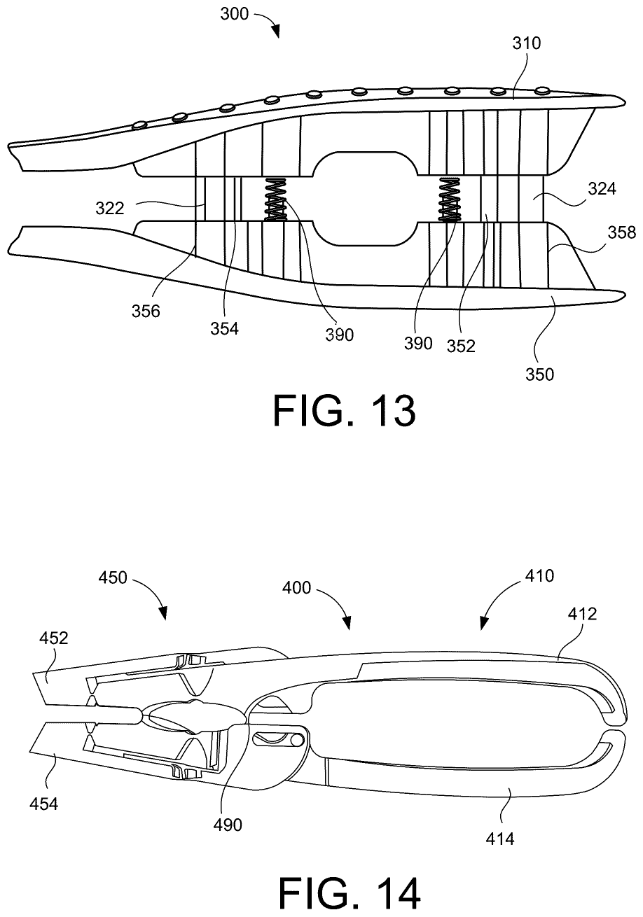

FIGS. 10-13 illustrate an apparatus 300 according to an aspect.

In some embodiments, that tool or apparatus 300 may be used to engage connectors that may be used to connect tubular member or tubes of a bodily implant. For example, in some embodiments, the tool 300 may be used to engage connectors used to connect tubular members of a penile implant such as a penile implant or a penile prosthesis.

The tool 300 includes a first elongate member 310 and a second elongate member 350. The first elongate member 310 includes a handle portion 312 and a distal portion 314. The second elongate member 350 includes a handle portion 352 and a distal portion 354.

In the illustrated embodiment, the distal portion 314 of the first elongate member 310 and the distal portion 354 of the second elongate member 350 are configured to engage a portion or portions of a bodily implant. For example, in some embodiments, the distal end portions are configured to engage connectors or portions of a connector that are used to connect tubular members of a bodily implant. In the illustrated embodiment, the distal end portion 314 and the distal end portion 354 each include a connector engagement portion.

The distal end portion 314 and 354 are configured to move with respect to each other. For example, in one embodiment, the distal end portions 314 and 354 are move a first configuration (or open configuration) to a second configuration (or closed configuration). The distance between the distal end portions 314 and 354 in the first configuration is greater than the distance between the distal end portions 314 and 354 when in the second configuration.

In the illustrated embodiment, the tool 300 includes a biasing member 390. The biasing member 390 is configured to bias the tool 300 such that the distal portions 314 and 354 is biased into one of the configurations. For example, in the illustrated embodiment, the biasing member 390 is configured to bias the distal portions 314 and 354 to an open configuration. In other embodiments, the biasing member may be configured to bias the distal portion to a closed configuration. In the illustrated embodiment, the tool 300 includes a first and second biasing members 390.

In the illustrated embodiment, the biasing member 390 is a spring member. In other embodiments, the biasing member is another type of biasing device or member.

In the illustrated embodiment, the first elongate member 310 includes a first projection 322 and a second projection 324. The second elongate member 350 includes a first receiving portion 356 that is configured to receive the first projection 322 of the first elongate member 310. The second elongate member 350 also includes a second receiving portion 358 that is configured to receive the second projection 324.

Similarly, the second elongate member 350 includes a first projection 322 and a second projection 324. The second elongate member 350 includes a first receiving portion 356 that is configured to receive the first projection 322 of the first elongate member 310. The second elongate member 350 also includes a second receiving portion 358 that is configured to receive the second projection 324.

In some embodiments, the tool 300 is configured to be a single use device. In other words, in some embodiments, the tool 300 is configured to be used in a single procedure on a single patient and then discarded or disposed of.

In some embodiments, the tool 300 is formed of a biocompatible plastic material. For example, in some embodiments, the first portion of the handle portion, the second portion of the handle portion, the first portion of the distal portion, and the second portion of the distal portion are formed or made from a biocompatible plastic material. In other embodiments, the tool 300 is formed of other materials including other biocompatible materials.

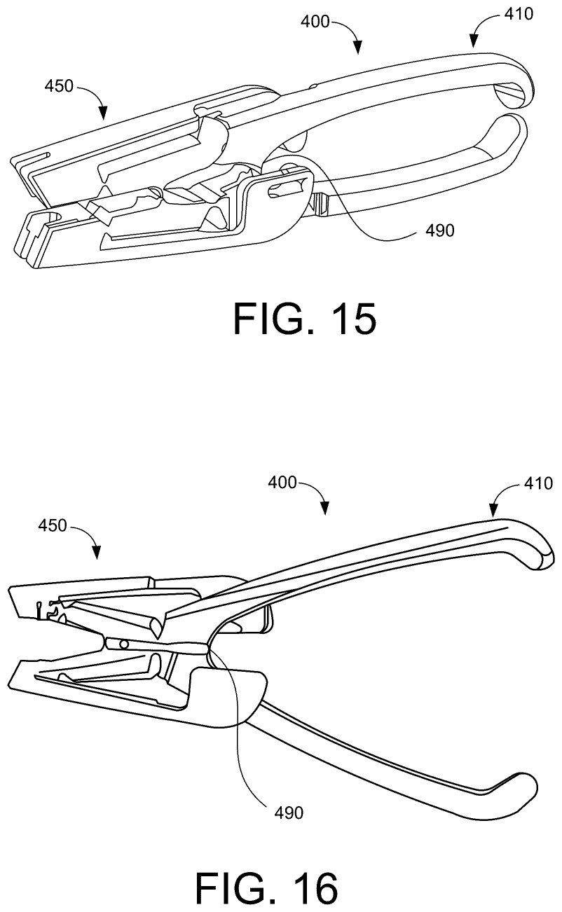

FIGS. 14-16 illustrate a tool 400 according to another aspect. The tool 400 may be used to engage connectors that may be used to connect tubular member or tubes of a bodily implant. For example, in some embodiments, the tool 400 may be used to engage connectors used to connect tubular members of a penile implant such as a penile implant or a penile prosthesis.

The tool 400 includes a handle portion 410 and a distal portion 450. The handle portion 410 includes a first portion 412 and a second portion 414. In some embodiments, the first portion 412 of the handle portion 410 is a separate member from the second portion 414 of the handle portion 410. In other embodiments, the first portion 412 and the second portion 414 may be formed unitarily or monolithically.

The distal portion 450 includes a first portion 452 and a second portion 454. In some embodiments, the distal portion 450 is configured to engage a portion of a bodily implant. For example, in some embodiments, the distal end portion 450 is configured to engage connectors that are used to connect tubular members of a bodily implant.

The first portion 452 of the distal portion 450 is configured to move with respect to the second portion 454 of the distal portion 450. For example, in one embodiment, the first portion 452 is configured to move with respect to the second portion 454 from a first configuration (or open configuration) to a second configuration (or closed configuration). The distance between the first portion 452 and the second portion 454 in the first configuration is greater than the distance between the first portion 452 and the second portion 454 when in the second configuration.

In the illustrated embodiment, the tool 400 includes a biasing member 490. The biasing member 490 is configured to bias the tool 400 such that the distal portion 450 is biased into one of its configurations. For example, in some embodiments, the biasing member 490 is configured to bias the distal portion 450 to an open configuration. In the illustrated embodiment, the biasing member 490 is a biased hinge such as a living hinge member.

The first portion 412 of the handle portion 410 is configured to move with respect to the second portion 414 of the handle portion 410. Movement of the first portion 412 with respect to the second portion 414 causes the first portion 452 of the distal portion 450 to move with respect to the second portion 454 of the distal portion 450. For example, movement of the first portion 412 of the handle portion 410 with respect to the second portion 414 of the handle portion 410 may cause the distal portion 450 to move from the first configuration (open configuration) to the second configuration (closed configuration).

In some embodiments, the tool 400 is configured to be a single use device. In other words, in some embodiments, the tool 400 is configured to be used in a single procedure on a single patient and then discarded or disposed of.

In some embodiments, the tool 400 is formed of a biocompatible plastic material. For example, in some embodiments, the first portion of the handle portion, the second portion of the handle portion, the first portion of the distal portion, and the second portion of the distal portion are formed or made from a biocompatible plastic material. In other embodiments, the tool 400 is formed of other materials including other biocompatible materials.

In use, the tool 400 may be placed within a body of a patient. The tool 400 may be placed such that the first portion 452 of the distal portion 450 engages a first portion of a connector and the second portion 454 of the distal portion 450 engages a second portion of the connector. The first portion 412 of the handle portion 410 may then be moved with respect to the second portion 414 of the handle portion 410 to move the distal portion from an open configuration to a closed configuration. The movement moves the first portion of the connector towards the second portion of the connector to connect the connector and any tubular members coupled to the connectors. The user may then release the handle portion to return the distal portion to the open configuration (because of the bias member).

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
a handle portion having a first portion and a second portion;
a distal portion disposed opposite the handle portion, the distal portion having a first connector contacting portion and a second connector contacting portion, the first connector contacting portion being configured to move with respect to the second connector contacting portion, the distal portion being configured to be disposed in a first configuration and a second configuration, the first connector contacting portion being disposed a first distance from the second connector contacting portion when the distal portion is in the first configuration, the first connector contacting portion being disposed a second distance from the second connector contacting portion when the distal portion is in the second configuration, the second distance being greater than the first distance; and
a biasing member configured to bias the distal portion into the first configuration.

2. The apparatus of claim 1, wherein the biasing member is disposed between the first portion of the handle portion and the second portion of the handle portion.

3. The apparatus of claim 1, wherein the biasing member is a spring.

4. The apparatus of claim 1, wherein the biasing member is a spring, the spring being configured to engage the first portion of the handle portion and the second portion of the handle portion.

5. The apparatus of claim 1, wherein the biasing member is a living hinge.

6. The apparatus of claim 1, wherein the first portion of the handle portion is configured to move with respect to the second portion of the handle portion.

7. The apparatus of claim 1, wherein the first portion of the handle portion being configured to move towards the second portion of the handle portion to place the distal portion in the second configuration.

8. The apparatus of claim 1, wherein the first portion of the handle portion includes a stop member, the second portion of the handle portion includes a stop member, the stop member of the first handle portion being configured to contact the stop member of the second handle portion.

9. The apparatus of claim 1, wherein the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location.

10. The apparatus of claim 1, wherein the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location, the first portion of the handle portion being coupled to the second portion of the handle portion.

11. The apparatus of claim 1, wherein the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location, the first portion of the handle portion being coupled to the second portion of the handle portion at a third location of the first portion of the handle portion, the third location being different than the first location, third location being different than the second location.

12. The apparatus of claim 1, wherein the distal portion has a first member and a second member, the first portion of the handle portion being coupled to the first member of the distal portion at a first location of the first portion of the handle portion, the first portion of the handle portion being coupled to the first member of the distal portion at a second location of the first portion of the handle portion, the first location being different than the second location, the first portion of the handle portion being coupled to the second portion of the handle portion at a third location of the first portion of the handle portion, the third location being disposed between the first location and the third location.

13. The apparatus of claim 1, wherein the first portion of the handle portion includes a projection portion, the second portion of the handle portion includes a receiving portion configured to receive the projection portion of the first portion of the handle portion.

14. The apparatus of claim 1, wherein the first portion of the handle portion includes a projection portion, the second portion of the handle portion includes a receiving portion configured to receive the projection portion of the first portion of the handle portion, the second portion of the handle portion includes a projection portion, the second portion of the handle portion includes a projection portion, the first portion of the handle portion includes a receiving portion configured to receive the projection portion of the second portion of the handle portion.

15. The apparatus of claim 1, wherein the first portion of the distal portion includes a receiving portion configured to receive a portion of a connector.

16. An apparatus, comprising:
a handle portion having a first member and a second member, the first member of the handle portion being coupled to the second member of the handle portion at a first location; and
a distal portion having a first member and a second member, the first member of the handle portion being coupled to the first member of the distal portion at a second location, the first member of the handle portion being coupled to the second member of the distal portion at a third location, the first location being disposed between the second location and the third location,
the first member of the distal portion having an engagement portion configured to engage a connector.

17. The apparatus of claim 16, further comprising a biasing member, the biasing member being disposed between the first member of the handle portion and the second member of the handle portion.

18. The apparatus of claim 16, wherein the first member of the distal portion is configured to move with respect to the second member of the distal portion from a first configuration to a second configuration.

19. An apparatus, comprising:
a first elongate member having a handle portion and a distal portion, the first elongate member having a projection;
a second elongate member having a handle portion and a distal portion, the second elongate member having a receiving portion, the receiving portion being configured to receive the projection; and
a biasing member disposed between the first elongate member and the second elongate member.

20. The apparatus of claim 19, wherein the first elongate member is configured to move with respect to the second elongate member from a first configuration to a second configuration.
